(19) United States
(12) Patent Application Publication
(10) Pub. No.: US 2007/0255337 A1
(43) Pub. Date: Nov. 1, 2007

(54) Cardiac monitoring via gastrointestinal stimulator

(75) Inventor: Richard M.T. Lu, Medina, MN (US)

Correspondence Address: SHUMAKER & SIEFFERT, P. A., 1625 RADIO DRIVE, SUITE 300, WOODBURY, MN, 55125, US

(73) Assignee: Medtronic, Inc.

(21) Appl. No.: 11/414,531

(22) Filed: Apr. 28, 2006

(51) Int. Cl. A61N 1/00

(52) U.S. Cl. 607/40

(57) ABSTRACT

The invention is directed to techniques for delivering electrical stimulation to a gastrointestinal tract of a patient via an implantable stimulator and monitoring electrical activity of a patient's heart by sensing cardiac signals via electrodes coupled to the implantable stimulator. The implantable stimulator may analyze the cardiac signals and generate a cardiac therapy recommendation for the patient, or may communicate the cardiac signals via telemetry circuitry to an external device for analysis. The sensed cardiac signals may be electrocardiogram (ECG) signals.

### TECHNICAL FIELD

The invention relates to implantable medical devices and, more particularly, to implantable gastrointestinal stimulators.

### BACKGROUND

Obesity is a serious health problem for many people. Patients who are overweight often have problems with mobility, sleep, high blood pressure, and high cholesterol. Some other serious risks associated with obesity include diabetes, cardiac arrest, stroke, kidney failure, and mortality. An obese person may be at a greater risk for developing cardiac problems.

Multiple factors contribute to obesity, including physical inactivity and overeating. Existing therapies include diet, exercise, appetite suppressive drugs, metabolism enhancing drugs, surgical restriction of the gastric tract, and surgical modification of the gastric tract. These therapies may result in little or no weight loss up to weight loss of nearly 50% of initial body weight.

Electrical stimulation of the gastrointestinal tract has been proposed to treat obesity, as well as to increase gastric motility and to treat symptoms of gastroparesis. For example, electrical stimulation of the gastrointestinal tract, and especially the stomach, is effective in suppressing symptoms of nausea and vomiting secondary to diabetic or idiopathic gastroparesis.

Typically, electrical stimulation involves the use of electrodes implanted in the wall of a target organ. The electrodes are electrically coupled to an implanted or external pulse generator via implanted or percutaneous leads. The pulse generator delivers a stimulation waveform via the leads and electrodes.

### SUMMARY

In general, the invention is directed to techniques for monitoring electrical activity of a patient's heart by sensing cardiac signals via an implantable stimulator that delivers stimulation pulses to the patient's gastrointestinal tract. The implantable stimulator may analyze the cardiac signals and generate a cardiac therapy recommendation for the patient, or communicate the cardiac signals via telemetry circuitry to an external device for analysis. The sensed cardiac signals may be electrocardiogram (ECG) signals.

In some embodiments, the cardiac signals may be transmitted to a central network server, such as a server of a patient management system. The server may present, via a web browser, web pages containing information, analysis, or recommendations to the patient or a physician, family member, friend, or caregiver of the patient. When analysis of the cardiac signals indicates a serious cardiac problem such as ventricular fibrillation or fast ventricular tachycardia, the implantable stimulator may initiate an alert procedure. For example, the implantable stimulator may issue an emergency communication that is relayed by an external module to an emergency response system to request immediate emergency assistance. In some embodiments, the patient may receive an alert, for example, from an implanted device, a wristwatch, a cellular telephone, or other device, that may advise the patient to seek immediate medical attention.

The techniques may provide an opportunity for early diagnosis of underlying cardiac problems or disease in patients implanted with gastric stimulators. The sensing and analysis of the cardiac signals may also provide a clinician an opportunity to identify patients for whom an implantable cardiac device would be beneficial. Moreover, the sensing and analysis of the cardiac signals may allow a patient or doctor to be alerted to a serious or life-threatening cardiac event, which may allow the patient to obtain prompt medical attention.

In one embodiment, the disclosure provides a method comprising delivering electrical stimulation to a gastrointestinal tract of a patient via an implantable stimulator, and sensing cardiac signals via a plurality of electrodes coupled to the implantable stimulator.

In another embodiment, the disclosure provides an implantable device comprising a plurality of electrodes, a pulse generator that delivers electrical stimulation to a gastrointestinal tract of a patient via at least some of the electrodes, and a processor that senses cardiac signals via at least some of the electrodes.

In a further embodiment, a system comprises an implantable device that delivers electrical stimulation to a gastrointestinal tract of a patient with a pulse generator and senses cardiac signals via a plurality of electrodes, and an external module, wherein the implantable device transmits information based on the cardiac signals to the external module via a telemetry interface associated with the implantable device.

In yet another embodiment, an implantable device comprises means for delivering electrical stimulation to a gastrointestinal tract of a patient via an implantable stimulator, and means for sensing cardiac signals via a plurality of electrodes coupled to the implantable stimulator.

In another embodiment, a computer-readable medium comprises instructions that cause a programmable processor to deliver electrical stimulation to a gastrointestinal tract of a patient via an implantable stimulator, and sense cardiac signals via a plurality of electrodes coupled to the implantable stimulator.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1C are schematic diagrams illustrating implantable stimulation systems with various configurations of ECG sensors.

FIG. 2 is a block diagram illustrating an implantable stimulator in greater detail in accordance with an embodiment of the invention.

FIG. 3 is a block diagram illustrating an example system in which a patient receives stimulation therapy, and information based on sensed ECG signals is communicated to the patient, a physician, or a family member.

FIG. 4 is a flowchart illustrating an example mode of operation of a processor in analyzing sensed electrocardiogram (ECG) data.

FIG. 5 is a flowchart illustrating an example mode of operation of a processor in detecting cardiac problems.

FIG. 6 is a timing diagram illustrating delivery of stimulation and sensing periods for sensing ECG data.

FIGS. 7A and 7B are exemplary screen illustrations depicting example reports showing ECG and stimulation information as viewed on a user interface.

### DETAILED DESCRIPTION

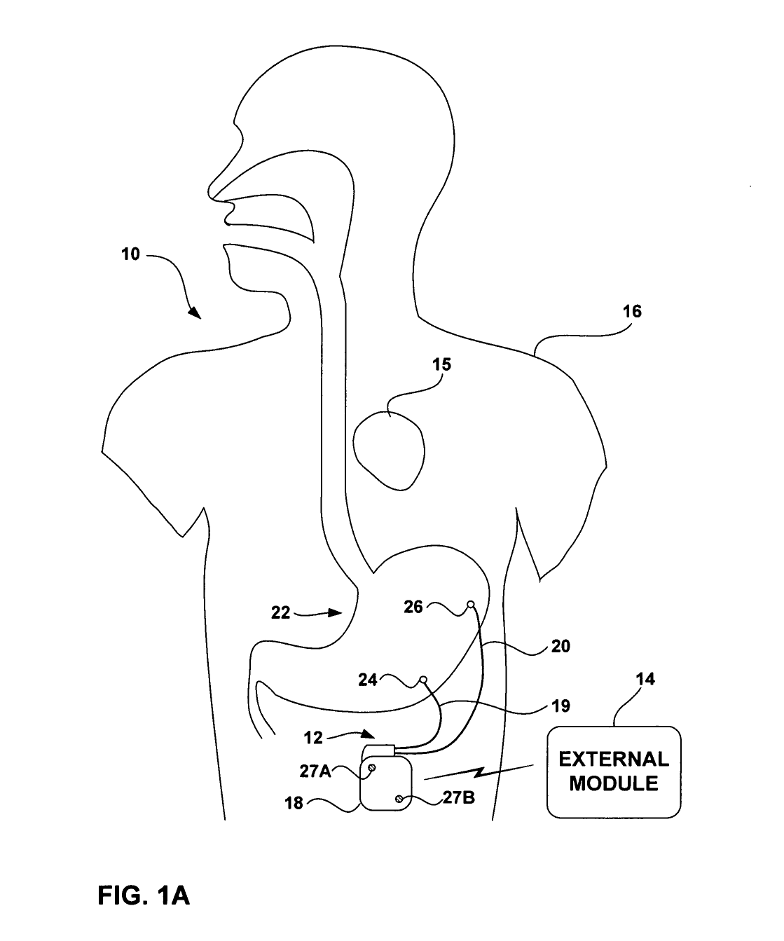

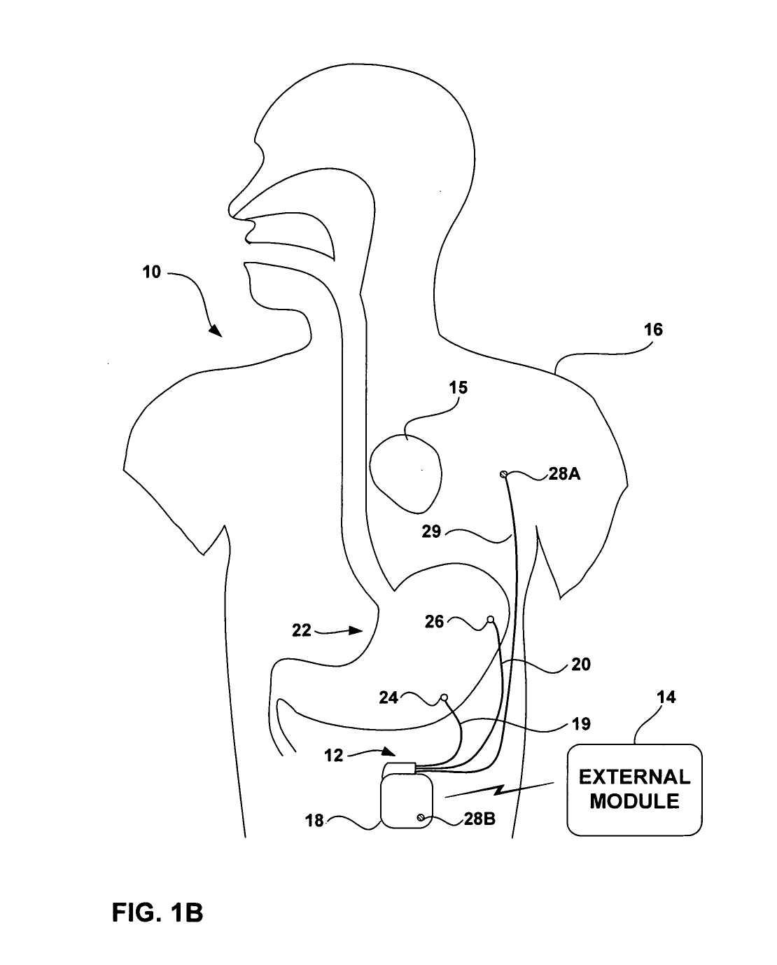

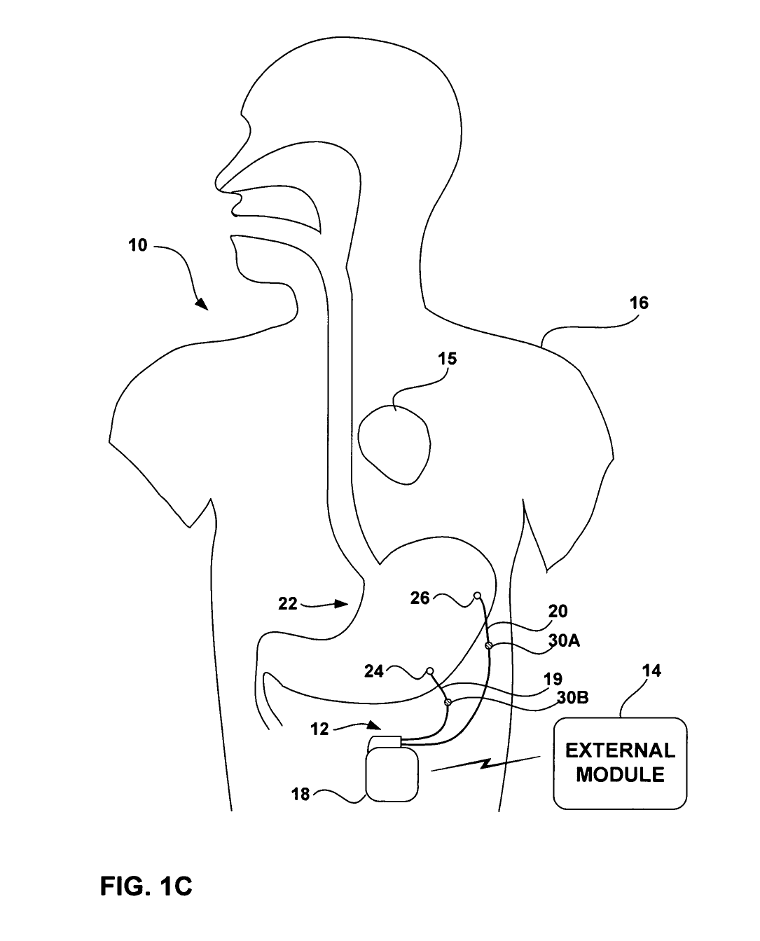

FIG. 1A is a schematic diagram illustrating an implantable stimulation system 10. System 10 is configured to provide gastric stimulation therapy, and is also configured to perform cardiac sensing and monitoring. As shown in FIG. 1A, system 10 includes an implantable gastric stimulator 12. Gastric stimulation therapy may be provided to treat obesity, gastroparesis, or other gastrointestinal disorders or diseases. The combination of gastric stimulation therapy and cardiac monitoring may be particularly valuable, because overweight or obese patients may also be at risk for developing cardiac problems. Hence, an implanted gastric stimulator provides a useful platform to monitor the patient for cardiac problems.

Cardiac signals are sensed by a plurality of electrodes attached to implantable stimulator 12. The signals may be electrocardiogram (ECG) signals that detect electrical activity of the heart 15 of patient 16. The sensed ECG signals are analyzed to determine whether the heart 15 or associated blood vessels of patient 16 are performing normally, or suffering from abnormalities, damage, or disease. Information obtained by analyzing the ECG signals of patient 16 may be used to provide alerts to patient 16 or a physician, or may be used to generate a cardiac therapy recommendation based on the analysis. For example, a clinician may recommend that patient 16 should be implanted with a cardiac device based on the analysis. In some embodiments, system 10 may be configured to control gastric stimulation parameters as a function of detected cardiac activity.

As further shown in FIG. 1A, system 10 may include an implantable stimulator 12 and external module 14 shown in conjunction with patient 16. Stimulator 12 includes a pulse generator 18 that generates electrical stimulation pulses. One or more leads 19, 20 carry the electrical stimulation pulses to stomach 22. Leads 19, 20 each include one or more electrodes 24, 26 for delivery of the electrical stimulation pulses to stomach 22. Although the electrical stimulation pulses may be delivered to other areas within the gastrointestinal tract, such as the esophagus, duodenum, small intestine, or large intestine, delivery of stimulation pulses to stomach 22 will generally be described in this disclosure for purposes of illustration.

The stimulation pulses may be configured to treat obesity by inducing sensations of nausea or satiety to reduce appetite and discourage overeating by the patient, and/or by increasing gastric motility to reduce caloric absorption by the patient. Although described for exemplary purposes in terms of a stimulator for treating obesity, the techniques of the invention may be applied more generally in the context of gastrointestinal stimulation for treatment of a variety of conditions. For example, stimulation pulses may be applied to regulate gastrointestinal motility, to treat symptoms of gastroparesis, or for treatment of other conditions. For example, electrical stimulation of the gastrointestinal tract, and especially the stomach, may be effective in suppressing symptoms of nausea and vomiting secondary to diabetic or idiopathic gastroparesis.

System 10 also includes sensing electrodes 27A, 27B for sensing electrical activity of the heart 15 of patient 16. In the exemplary embodiment of FIG. 1, electrodes 27A, 27B are provided on the housing of implantable stimulator 12. The spacing of electrodes 27A and 27B may be on the order of about one inch (2.5 cm) but can be larger or smaller depending on the exact size of the implantable stimulator 12. Smaller devices and closer spacing may require greater amplification of the detected cardiac signals.

Implantable stimulator 12 receives cardiac signals sensed by electrodes 27A, 27B and stores information based on the cardiac signals in memory associated with the implantable stimulator. For example, implantable stimulator 12 may store an electrocardiogram (ECG) generated based on the sensed cardiac signals. In a typical implementation, the ECG represents a difference in potential between two or more electrodes placed upon or within the body of the patient. A processor within implantable stimulator 12 detects the ECG signals associated with the contraction of the heart 15 and amplifies the ECG signals so that the ECG signals can be analyzed and/or displayed for analysis.

Sensing of ECG signals may occur continuously, periodically, or intermittently, as therapy dictates. For example, sensing intervals may alternate with intervals of gastric stimulation. As another example, sensing intervals and gastric intervals may overlap or occur simultaneously. Information relating to the sensed data may be stored in memory within pulse generator 18 for retrieval and analysis at a later time. Alternatively, the sensed data may be immediately transmitted to external module 14 by wired or wireless telemetry, e.g., as raw sensed data or pre-processed data indicating ECG signals or particular cardiac events.

At the surface lining of stomach 22, leads 19, 20 penetrate into tissue such that electrodes 24 and 26 are positioned to deliver stimulation to the stomach. The stimulation pulses generated by stimulator 12 may cause the smooth muscle of stomach 22 to contract and slowly move contents from the entrance toward the exit of the stomach. Alternatively, or additionally, the electrical stimulation pulses may stimulate nerves within stomach 22 to cause muscle contraction and thereby restore or enhance gastrointestinal motility. Alternatively, as mentioned above, the stimulation pulses may be applied to induce nausea or satiety in response to monitored parameters. The induced sensation of nausea or satiety may reduce a patient's desire to consume large portions of food. Enhanced motility may serve to speed food through the gastrointestinal tract and reduce caloric absorption. Again, the stimulation pulses may be delivered elsewhere within the gastrointestinal tract, either as an alternative to stimulation of stomach 22 or in conjunction with stimulation of the stomach.

Implantable stimulator 12 may be constructed with a biocompatible housing, such as titanium, stainless steel, or a polymeric material, and is surgically implanted within patient 16. Although illustrated as being implanted near stomach 22 of patient 16, implantable stimulator 12 may alternatively be implanted in other areas of patient 16, such as the upper thorax, chest area, or buttocks. The implantation site may be a subcutaneous location in the side of the lower abdomen or the side of the lower back, or other appropriate site. Pulse generator 18 is housed within the biocompatible housing, and includes components suitable for generation of electrical stimulation pulses. Electrical leads 19 and 20 are flexible, electrically insulated from body tissues, and terminated with electrodes 24 and 26 at the distal ends of the respective leads. The leads may be surgically or percutaneously tunneled to stimulation sites on stomach 22. The proximal ends of leads 19 and 20 are electrically coupled to pulse generator 18 via internal conductors to conduct the stimulation pulses to stomach 22 via electrodes 24, 26.

Leads 19, 20 may be placed into the muscle layer or layers of stomach 22 via an open surgical procedure, or by laparoscopic surgery. Leads also may be placed in the mucosa or submucosa by endoscopic techniques, or by an open surgical procedure or laparoscopic surgery. Electrodes 24, 26 may form a bipolar pair of electrodes. Alternatively, pulse generator 18 may carry a reference electrode to form an “active can” arrangement, in which one or both of electrodes 24, 26 are unipolar electrodes referenced to an electrode on the pulse generator housing. A variety of polarities and electrode arrangements may be used.

The stimulation pulses delivered by implantable stimulator 12 are characterized by stimulation parameters such as a voltage or current amplitude, pulse width, and pulse rate. In addition, in some embodiments, the stimulation parameters may include electrode combination and polarity that specify different combinations of electrodes, if available, and polarities of the electrodes as anodes or cathodes. The stimulation parameters may be fixed, adjusted in response to sensed physiological conditions within or near stomach 22, or adjusted in response to patient input entered via external module 14. For example, in some embodiments, patient 16 may be permitted to adjust stimulation amplitude and turn stimulation on and off using external module 14.

As an illustration, the stimulation pulses delivered by stimulator 12 may have a pulse amplitude in a range of approximately 1 to 10 volts, a pulse width in a range of approximately 50 microseconds to 10 milliseconds, and a pulse rate in a range of approximately 1 to 100 Hz. The pulse rate is more preferably in a range of approximately 2 to 40 Hz, and even more preferably in a range of approximately 5 to 20 Hz. The terms pulse rate and pulse frequency may be used interchangeably in this description. In some embodiments, an instant start to delivery of the stimulation pulses may be provided. However, a gradual ramp up in stimulation intensity may be applied to prevent muscle shock and patient discomfort. This ramp may be in the form of a gradually increasing pulse rate, amplitude, or pulse width.

Stimulator 12 also may include telemetry electronics to communicate with external module 14. External module 14 may be a small, battery-powered, portable device that accompanies patient 16 throughout a daily routine. External module 14 may have a simple user interface, such as a button or keypad, and a display or lights. External module 14 may be a hand-held device configured to permit activation of stimulation and adjustment of stimulation parameters. Alternatively, external module 14 may form part of a larger device including a more complete set of programming features including complete parameter modifications, firmware upgrades, data recovery, or battery recharging in the event stimulator 12 includes a rechargeable battery. External module 14 may be a patient programmer, a physician programmer, or a patient monitor. In some embodiments, external module 14 may be a general purpose device such as a cellular telephone, a wristwatch, a personal digital assistant (PDA), or a pager.

In some example embodiments, implantable stimulator 12 may communicate the sensed ECG signals to external module 14. The communication may occur wirelessly, or in the case of a percutaneous lead implantable stimulator 12 may have a wired connection. However, in most cases in which implantable stimulator 12 is fully implanted, communication between implantable stimulator 12 and external module 14 will occur wirelessly. Communication may occur continuously, periodically, or intermittently. External module 14 may analyze the ECG signals. Alternatively, external module 14 may transmit the received ECG signals to another device for analysis, such as a central server accessed by external module 14 via the Internet. In other embodiments, implantable stimulator 12 may include a processor that performs analysis of the ECG signals, and communicates information obtained based on the analysis to external module 14. Accordingly, the computing resources for analysis of ECG signals may be provided within stimulator 12, external module 14, or elsewhere.

Analysis of the ECG signals may be performed to detect a variety of cardiac conditions, such as atrial fibrillation, ventricular fibrillation, atrial tachycardia, ventricular tachycardia, bradycardia, wide QRS complexes due to unsynchronized ventricular contractions, ectopic events, ischemic events, asystole, ST elevation, premature atrial contraction, bundle branch block, myocardial infarction, irregular heart rate, long QT syndrome, or other heart conditions detectable by analyzing ECG signals. In some embodiments, an activity sensor or minute ventilation sensor may also be included in conjunction with implantable stimulator. For example, an activity sensor (e.g., an accelerometer) or minute ventilation sensor may be disposed within the housing of implantable stimulator 12, or attached to implantable stimulator via a lead. In one embodiment, sensing electrodes 27 may be used to simply sense heart rate. For example, data sensed by the activity sensor or minute ventilation sensor may be used with the heart rate or ECG signals to detect conditions such as chronotropic incompetence.

External module 14 may present feedback to patient 16 regarding ECG signals. Alternatively, such feedback may be presented by a central server, e.g., via a web page, to patient 16, or a caregiver, family member, or health service provider of patient 16. Stimulator 12 may provide an alert to patient 16 to indicate, for example, that a serious cardiac event has occurred or is occurring. Stimulator 12 may adjust stimulation therapy in response to the ECG signals. For example, stimulator may increase or decrease the level or duration of stimulation.

In some embodiments, system 10 may include multiple implantable stimulators 12 to stimulate a variety of regions of stomach 22. Stimulation delivered by the multiple stimulators may be coordinated in a synchronized manner, or performed without communication between stimulators. Also, the electrodes may be located in a variety of sites on the stomach, or elsewhere in the gastrointestinal tract, dependent on the particular therapy or the condition of patient 12.

The electrodes carried at the distal end of each lead 19, 20 may be attached to the wall of stomach 22 in a variety of ways. For example, the electrode may be surgically sutured onto the outer wall of stomach 22 or fixed by penetration of anchoring devices, such as hooks, barbs or helical structures, within the tissue of stomach 22. Also, surgical adhesives may be used to attach the electrodes. In any event, each electrode is implanted in acceptable electrical contact with the smooth muscle cells within the wall of stomach 22. In some cases, the electrodes may be placed on the serosal surface of stomach 22, within the muscle wall of the stomach, or within the mucosal or submucosal region of the stomach.

FIG. 1B is a schematic diagram illustrating implantable stimulation system 10 in which another exemplary arrangement of sensing electrodes is employed. As shown in FIG. 1B, an electrode 28A extends away from the implantable stimulator 12 via lead 29. In the example of FIG. 1B, lead 29 is a dedicated lead provided for cardiac sensing, and is provided in addition to gastric leads 19, 20. Another electrode 28B is positioned on the housing of implantable stimulator 12. Electrodes 28A, 28B are dedicated to sensing of ECG signals. The configuration of FIG. 1B may achieve a greater inter-electrode spacing than other configurations. In some embodiments, electrode 28A and lead 29 may be positioned intravenously. Alternatively, electrode 28A may be formed as a subcutaneous patch and placed anywhere in the thorax of patient, but more preferably in the upper chest region near heart 15.

FIG. 1C is a schematic diagram illustrating an implantable stimulation system 10 in which yet another exemplary arrangement of sensing electrodes is employed. As shown in FIG. 1C, one or both of leads 19, 20 may carry a sense electrode 30A, 30B, in addition to stimulation electrodes 24, 26, to sense ECG signals. Sense electrodes 30A, 30B may be positioned at other areas on leads 19, 20 than the areas shown.

The electrode configurations of the devices shown in FIGS. 1A-1C are exemplary only, and other combinations and configurations of electrodes may be used. For example, one or more electrodes may be located on an edge of the housing of implantable stimulator 12. The electrodes placed on the edge of implantable stimulator 12 may constitute insulated pins of feedthroughs extending through the housing. The housing of implantable stimulator 12 may include the “can” as well as a header on the can. As another example, a plurality of electrodes may be provided as an array of electrodes. As yet another example, sensing may be performed via stimulation electrodes 24, 26. In this case, electrodes 24, 26 perform the dual role of stimulation and sensing, e.g., on an alternating basis. Although FIGS. 1A-1C illustrate an implantable stimulator including two sense electrodes, more than two sense electrodes may be provided. In one embodiment, four or more electrodes may be coupled to the stimulator or adjacent to the stimulator, and a physician may select which of the electrodes will be activated for a given patient.

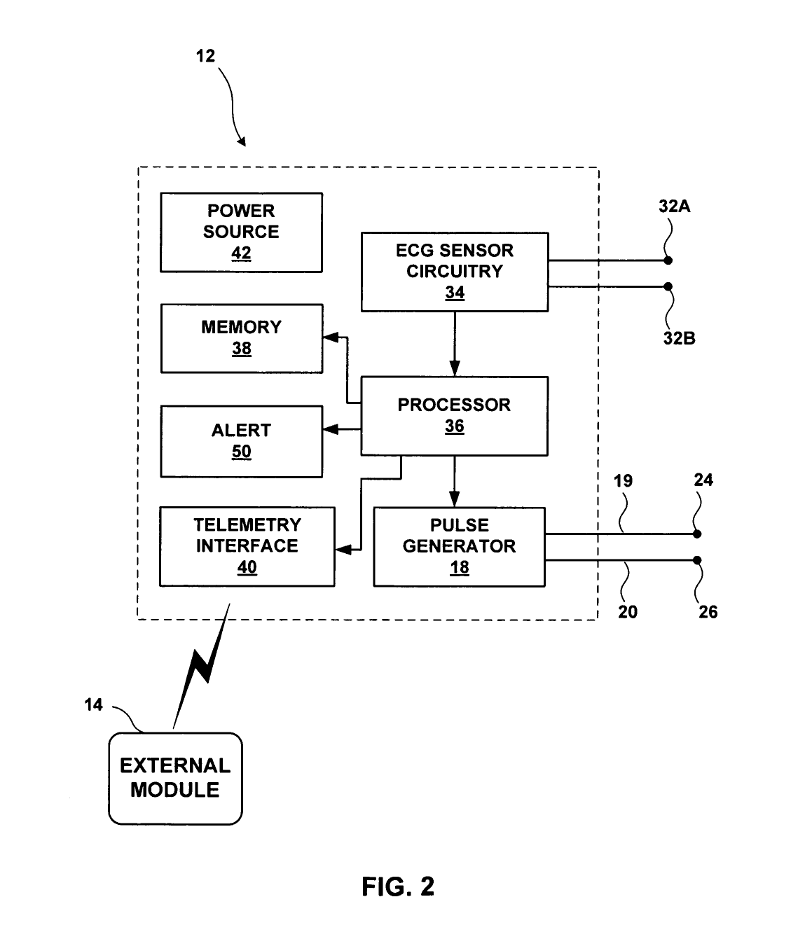

FIG. 2 is a block diagram illustrating various components of implantable stimulator 12 in greater detail in accordance with an embodiment of the invention. In FIG. 2, implantable stimulator 12 includes ECG sensors 32A and 32B (“ECG sensors 32”). ECG sensors 32A and 32B may be configured in any of the arrangements described above with respect to FIGS. 1A-1C. In some embodiments, implantable stimulator 12 may include more than two ECG sensors. Signals detected by ECG sensors 32 may be representative of electrical activity of the heart 15 of patient 16. ECG sensor circuitry 34 receives the sensed ECG signals, and supplies the ECG signals to a processor 36. ECG sensor circuitry 34 may amplify and filter the ECG signals to condition the signals before supplying the signals to processor 36.

Processor 36 processes the received ECG signals, and may analyze the ECG signals. The received ECG signal is typically converted to digital values prior to processing by processor 36, and stored in memory 38. For example, processor 36 may analyze the ECG signals to provide diagnostic information used to determine the onset of an arrhythmia or other cardiac conditions. Processor 36 may also determine a type of arrhythmia or other cardiac problem by analyzing the rate and morphology of the sensed cardiac signals. For example, processor 36 may analyze heart rate variability and features of the ECG signals such as the P wave, QRS complex, T wave, QT interval, PR interval, ST segment, or other features.

As examples, irregular or absent P waves may signify arrhythmia, while the shape of the P waves may be a sign of atrial problems. Very wide and deep Q waves may signify myocardial infarction. Abnormalities in the QRS complex may be a sign of bundle branch block, ventricular origin of tachycardia, ventricular hypertrophy or other ventricular abnormalities. T wave abnormalities may signify electrolyte disturbance, such as hyperkalemia and hypokalemia. Upward or downward displacement of the ST segment may indicate damage to the cardiac muscle or strain on the ventricles. The ST segment can be depressed or elevated depending on the ECG vector in myocardial infarction or ischemia.

ECG signal characteristics indicative of cardiac problems are well known to those skilled in the art of cardiology. Accordingly, the possible ECG signal characteristics described above are for purposes of illustration and should not be considered limiting of the invention as broadly embodied and described herein. Rather, the invention may be applied to obtain and analyze any of a variety of ECG signal characteristics obtained by an implantable gastrointestinal stimulator to identify cardiac problems that may indicate the need for immediate cardiac care or referral for possible cardiac therapy.

Memory 38 may include any form of volatile memory, non-volatile memory, or both. In addition to data sensed via ECG sensors 32, memory 38 may store records concerning measurements of sensed ECG signals, communications to patient 16, or other information pertaining to operation of implantable stimulator 12. Memory 38 may also store information about patient 16 and stimulation therapy parameters. In addition, processor 36 is typically programmable, and programmed instructions reside in memory 38.

Wireless telemetry in stimulator 12 may be accomplished by radio frequency (RF) communication or proximal inductive interaction of implantable stimulator 12 with external module 14 via telemetry interface 40. Processor 36 controls telemetry interface 40 to exchange information with external module 14. Processor 36 may transmit operational information and sensed information to external module 14 via telemetry interface 40. For example, processor 36 may transmit sensed ECG signals, or other information relating to analysis of ECG signals. Also, in some embodiments, pulse generator 18 may communicate with other implanted devices, such as stimulators or sensors, via telemetry interface 40.

Power source 42 delivers operating power to the components of implantable stimulator 12. Power source 42 may include a battery and a power generation circuit to produce the operating power. In some embodiments, the battery may be rechargeable to allow extended operation. Recharging may be accomplished through proximal inductive interaction between an external charger and an inductive charging coil within implantable stimulator 12. In other embodiments, an external inductive power supply may transcutaneously power implantable stimulator 12 whenever stimulation therapy is to occur.

Implantable stimulator 12 is coupled to electrodes 24, 26 by leads 19, 20, respectively. Implantable stimulator 12 provides stimulation therapy to the gastrointestinal tract of patient 16. Pulse generator 18 includes suitable pulse generation circuitry for generating a voltage or current waveform with a selected amplitude, pulse width, and frequency. In some embodiments, processor 36 may determine whether to direct application of electrical stimulation to patient 16 and/or adjust stimulation parameters based upon sensed ECG data. Alternatively, or additionally, processor 36 may be responsive to instructions from external module 14 to direct application of electrical stimulation and/or adjust stimulation parameters. Processor 36 may include at least one programmable timing counter (not shown) that is used to measure timing intervals. For example, the timing counter may measure timing intervals of sensing intervals or stimulation intervals.

Processor 36 may also record the occurrence of electrical stimulation within memory 38 for use in determining whether additional electrical stimulation is desired to increase an amount of negative biofeedback provided to the patient 16. For example, processor 36 stores an occurrence of electrical stimulation in memory 38. The next time processor 36 determines electrical stimulation is needed, processor 36 may search memory 38 to determine when the prior electrical stimulation occurred in order to estimate whether electrical stimulation for an extended period of time may be useful.

If a patient 16 consumes food on more occasions or for longer durations than may be specified in a particular treatment plan for obesity, electrical stimulation for extended periods of time beyond a baseline time period may be useful to encourage patients to reduce the duration or number of occasions in which food is consumed. Similarly, a record of the prior occurrence of electrical stimulation may be used to ensure that a minimum amount of time passes between the detection of gastric activity. When gastric activity is detected before the minimum amount of time has passed, electrical stimulation may also be provided for an extended period of time to discourage patient 16 from eating food as often.

In embodiments where processor 36 analyzes ECG signals, processor 36 may communicate results of the analysis to patient 16 in a number of ways. Implantable stimulator 16 may wirelessly transmit information to external module 14 using telemetry interface 40. External module 14 may notify patient 16 when the ECG signals indicate a cardiac problem. External module 14 may notify patient 16 in the form of a visible or audible notification, e.g., emitted by a light, LED, display, or audio speaker. A visible notification may be presented as text, graphics, one or more blinking lights, illumination of one or more lights, or the like. An audible notification may take the form of an audible beep, ring, speech message, or the like. In addition to transmitting a communication to an external module 14, telemetry interface 40 may be configured to wirelessly transmit information about the history or status of implantable stimulator 12 to a physician for patient 16.

In addition, or in the alternative, implantable stimulator 12 may include an alert module 50 that is implanted in the body of patient 16. When activated by processor 36, alert module 50 can notify patient 16 directly without use of external module 14. Alert module 50 may, for example, notify patient 16 audibly or by vibration. For example, alert module 50 may take the form of a piezoelectric transducer that is energized in response to a signal from processor 36 in order to emit a sound or vibration. Alternatively, alert module 50 may apply electrical stimulation to the patient 16 at a level or in a pattern that is noticeable. In each case, patient 16 may receive a communication that implantable stimulator 12 has detected a serious cardiac event or condition. The communication may mean that patient 16 must seek immediate medical attention.

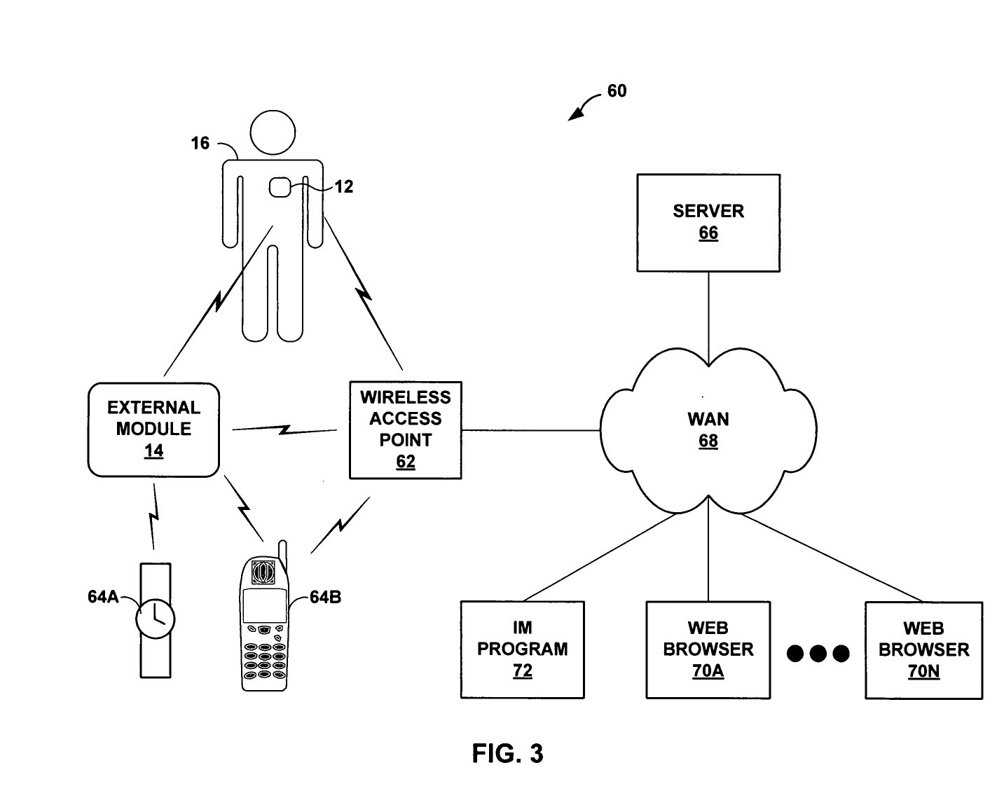

FIG. 3 is a block diagram illustrating an example system 60 in which patient 16 receives stimulation therapy, and information based on sensed ECG signals is communicated to patient 16, a call center, a physician, or a family member. As shown in FIG. 3, implantable stimulator 12 communicates wirelessly with external module 14 via radio frequency (RF) telemetry, but the communication may also be transmitted via a wired connection, an optical connection, or a transcutaneous communication link. External module 14 may be a patient programmer, i.e., a device dedicated to receiving user input pertaining to electric stimulation and transmitting corresponding commands to implantable stimulator 12. Implantable stimulator 12 may be interrogated by, or may voluntarily transmit information to, external module 14. As discussed above, the information obtained from implantable stimulator 12 may be preprocessed by implantable stimulator 12, processed by external module 14, or both.

As shown, external module 14 may communicate with general purpose devices 64A, 64B. In the illustrated example, external module 14 communicates with general purpose devices including a wristwatch 64A and a cellular telephone 64B. In other examples, external module 14 may communicate with a pager, personal digital assistant (PDA), or other general purpose device (not shown), which may be carried by patient 16. General purpose devices 64 may display text or graphical indications to patient 16. In some embodiments, external module 14 may itself be a general purpose device such as a pager, cellular telephone, or PDA.

External module 14 may transfer information to a docking station (not shown) upon being placed in the docking station. In other embodiments, external module 14 may wirelessly transfer data to wireless access point (WAP) 62. Alternatively, implantable stimulator 12 may communicate directly with WAP 62. WAP 62 may communicate information to cellular telephone 64B. In some embodiments, WAP 62 may transfer information to a server 66 via wide area network (WAN) 68. Server 66 may be a central server of a patient management system, and WAN 68 may be the Internet.

Server 66 may present web pages containing information via web browsers 70A-70N (“web browsers 70”) to users such as patient 16, or a doctor, family member, friend, or caregiver of patient 16. Server 66 may also present information via an instant message (IM) program 72 to patient 16 or other user. Patient 16 may view the information presented by web browser 70A and IM program 72 on a home computer. For example, server 66 may cause patient 16 to receive an alert via wristwatch 64A, cellular telephone 64B, or IM program 72 that instructs patient 16 to seek medical attention when patient 16 has an ECG that indicates a serious cardiac condition.

In some embodiments, some or all of the analysis operations discussed above with respect to processor 36 of FIG. 2 may be performed externally to implantable stimulator 12, such as within external module 14 or within server 66. For example, processor 36 of implantable stimulator 12 may collect ECG data and communicate the collected ECG data to external module 14 via telemetry interface 40. External module 14 may process and analyze the data, or may send the data to server 66 for processing and analysis.

In another embodiment, processor 36 may provide some processing of the data, and external module 14 or server 66 may provide additional processing and analysis. For example, processor 36 may determine information relating to an ECG signal, such as characteristics of the P wave, QRS complex, T wave, QT interval, PR interval, ST segment, and provide this information to external module 14. External module 14 or server 66 may then make a diagnosis or recommendation based on the information. Alternatively, external module or server 66 may present the information to a clinician, who makes a diagnosis or recommendation based on the information. For example, the clinician may recommend that patient 16 be implanted with a cardiac device based on the information.

Certain information obtained based on the ECG signals may be presented to patient 16 and/or other users (e.g., a doctor, family member, or caregiver of patient 16) by any of external module 14, devices 64, web browsers 70 or IM program 72. The information may relate to the analysis of ECG signals, such as whether the patient's heart 15 is functioning normally. Information may be presented via visible or audible output media provided by external module 14, such as lights, LEDs, a display or an audio speaker. An audio message may take the form of an audible beep, ring, speech message or the like. The patient 16, physician, family members, or other caregivers may use the information to take action, such as seeking medical attention for patient 16, or determining that patient 16 is a candidate for an implanted cardiac device or other cardiac therapy.

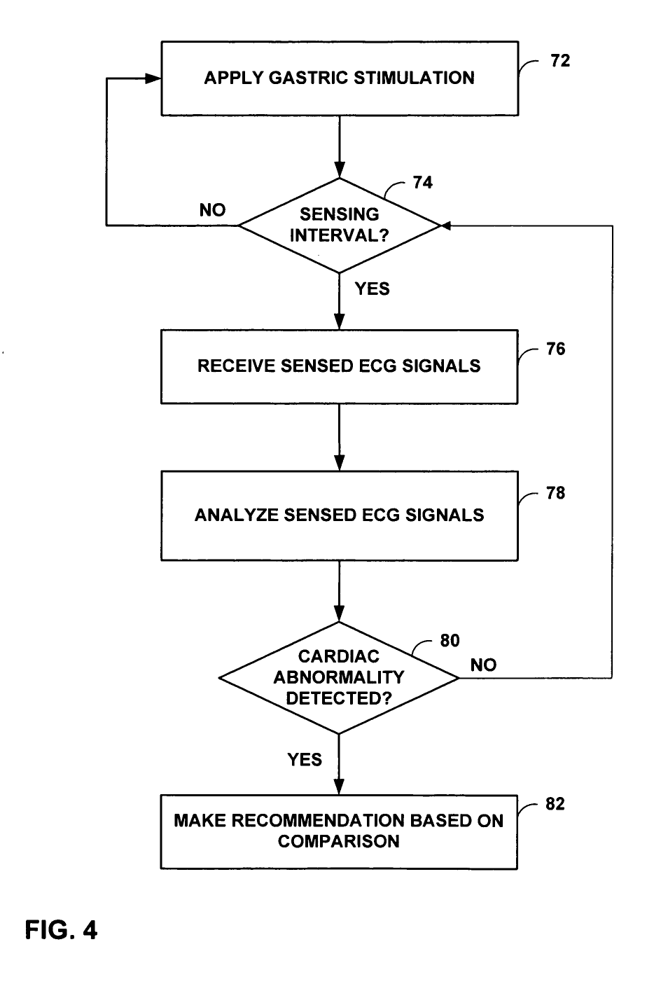

FIG. 4 is a flowchart illustrating an example mode of operation of processor 36 in analyzing sensed electrocardiogram (ECG) data. Processor 36 causes pulse generator 18 to apply electrical stimulation to stomach 22 via electrodes 24 and 26 (FIG. 1A) (72). If processor 36 determines that processor 36 is within a sensing interval (for example, based on a programmable timing counter that measures timing intervals) (YES branch of 74), processor 36 receives ECG signals sensed by sensors 32 (FIG. 2) (76). If processor 36 is not within a sensing interval (NO branch of 74), processor 36 will continue to apply gastric stimulation. In some embodiments, implantable stimulator 12 may stop applying gastric stimulation during sensing intervals. In other embodiments, sensing may occur continuously at all times or for extended periods at selected times of day.

Processor 36 may analyze the sensed ECG signals (78), or as described above, processor 36 may communicate the ECG signals to external module 14 for analysis. If processor 36 detects a cardiac abnormality (80), such as an arrhythmia or other condition, processor 36 may make a recommendation based on the comparison (82). In a situation where processor 36 detects a cardiac abnormality (that is not immediately life threatening), processor 36 may communicate to a clinician via external module 14 and server 66 a recommendation that patient 16 be implanted with an implantable cardiac device or undergo other clinical procedures such as atrial ablation or stenting. Therapy controller 58 may also cause a list of suggested actions to be displayed to patient 16, such as to lower cholesterol, eat more healthfully, or exercise more. Alternatively or additionally, therapy controller 58 may modify stimulation therapy parameters in response to the analysis. Processor 36 may perform some or all of the above steps hourly, daily, on demand, or at other time interval as configured by a user.

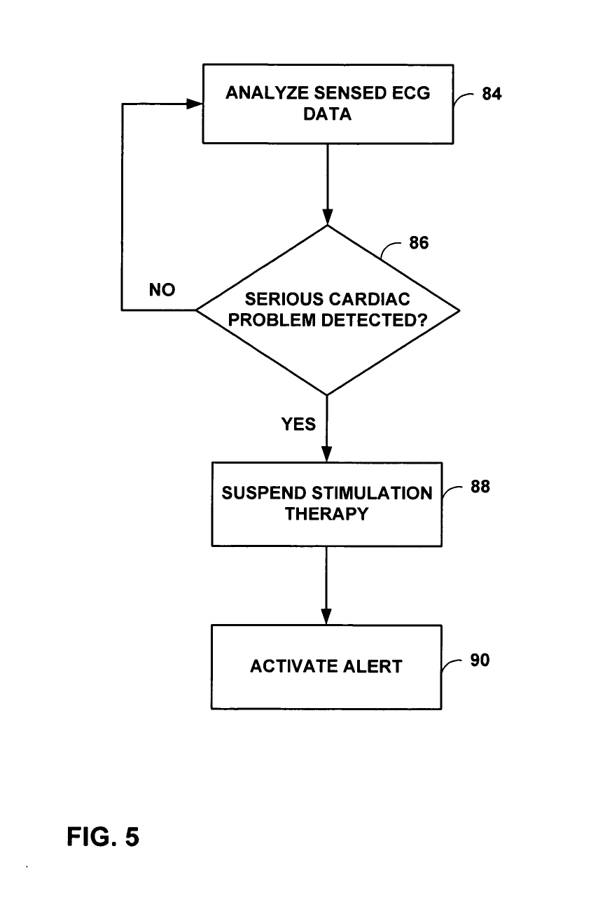

FIG. 5 is a flowchart illustrating another example mode of operation of a processor in detecting cardiac problems. Processor 36 analyses the sensed ECG signals (84). If processor 36 detects a serious cardiac problem based on the analysis (e.g., ventricular fibrillation or fast ventricular tachycardia) (86), processor 36 may completely suspend stimulation therapy by implantable stimulator 12 until further notice (88). For example, where implantable stimulator 12 alternates stimulation intervals with sensing intervals, if processor 36 detects a serious cardiac problem during a sensing interval, even when the sensing interval ends and the next stimulation interval begins, processor 36 may suspend stimulation therapy, and in some cases, may continue sensing beyond the proscribed sensing interval.

Processor 36 may be configured with particular parameters that, if detected, constitute a serious problem, which may trigger the steps shown in FIG. 5. For example, processor 36 may follow the steps when patient 16 is experiencing ventricular fibrillation or fast ventricular tachycardia. In response to the detection of a serious cardiac problem, processor 36 may also initiate an alert procedure (90). For example, processor 36 may issue a communication to an emergency response system via external module 14. The communication may request an immediate emergency response by a paramedic or other emergency responder. In some embodiments, implantable stimulator 12 may include or communicate with a global positioning system (GPS) unit that allows processor 36 to report the location of patient 16 to the emergency response system. Processor 36 may also issue an emergency communication that is relayed by external module 14 to a doctor via server 66.

As another example, processor 36 may also activate alert module 50 to issue an alert to patient 16. Alert module 50 may, for example, notify patient 16 audibly or by vibration. Alternatively, alert module 50 may apply electrical stimulation to the patient 16 at a level or in a pattern that is noticeable. In each case, patient 16 may receive a communication that implantable stimulator 12 has detected a serious cardiac event or condition. The communication may mean that patient 16 must seek immediate medical attention. Processor 36 may perform some or all of the above steps hourly, daily, on demand, or at other time interval as configured by a user.

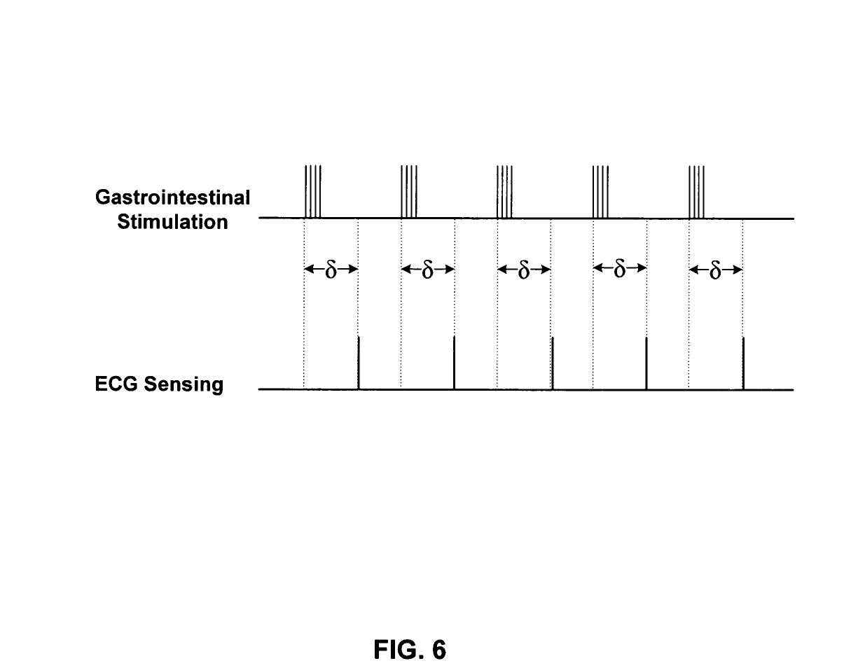

FIG. 6 is a timing diagram illustrating one example embodiment in which gastric stimulation periods alternate with sensing periods for sensing ECG data. As shown in FIG. 6, the stimulation pulses delivered by implantable stimulator 12 to stomach 22 are delivered as a series of pulse bursts. Each burst is characterized by a pulse rate for pulses delivered within the burst, a burst rate, and a burst length. For example, the stimulation pulses delivered by stimulator 12 may have a pulse amplitude in a range of approximately 1 to 10 volts, a pulse width in a range of approximately 50 microseconds to 10 milliseconds, and a pulse rate in a range of approximately 1 to 100 Hz. The pulse rate is more preferably in a range of approximately 2 to 40 Hz, and even more preferably in a range of approximately 5 to 20 Hz.

In the example of FIG. 6, each burst contains pulses delivered at a rate of approximately 40 Hz. The burst rate may be in a range of approximately 3 to 15 bursts per minute, which is approximately 1 to 5 times the typical gastric slow wave frequency in a healthy patient. The burst length may be in a range of approximately 10 to 50 percent of the burst period, i.e., the period between successive bursts. In the example of FIG. 6, the bursts delivered to stomach 22 are synchronized to alternate with sensing intervals. In particular, there is a time delay δ between the delivery of each burst of gastric stimulation and the start of a sensing period or interval. The term “sensing period” may refer to extended periods of time in which sensing occurs continuously. In some embodiments, sensing periods may overlap with stimulation periods. In other embodiments, sensing may occur continuously at all times. In these embodiments, processor 36 may filter out stimulation pulses from the sensed data.

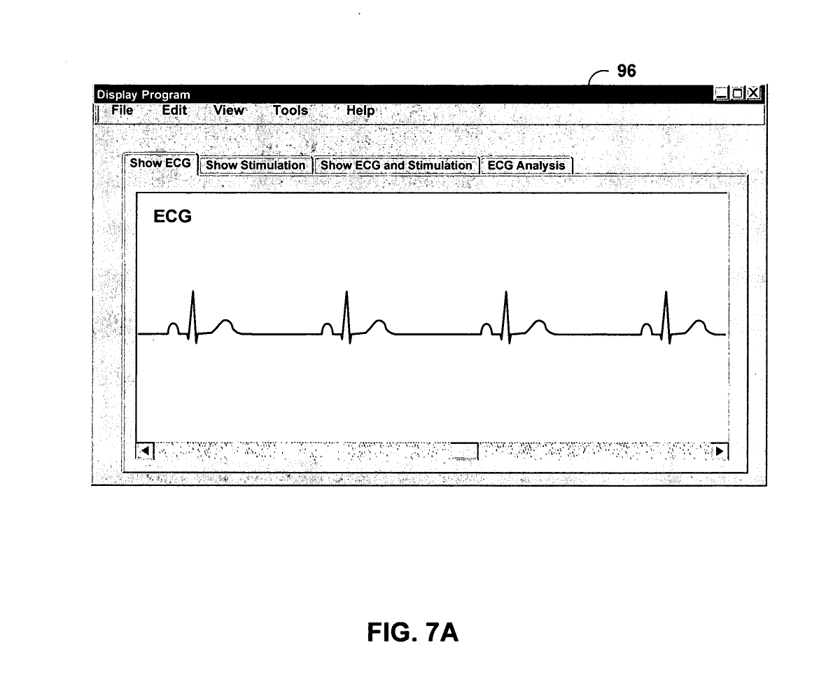

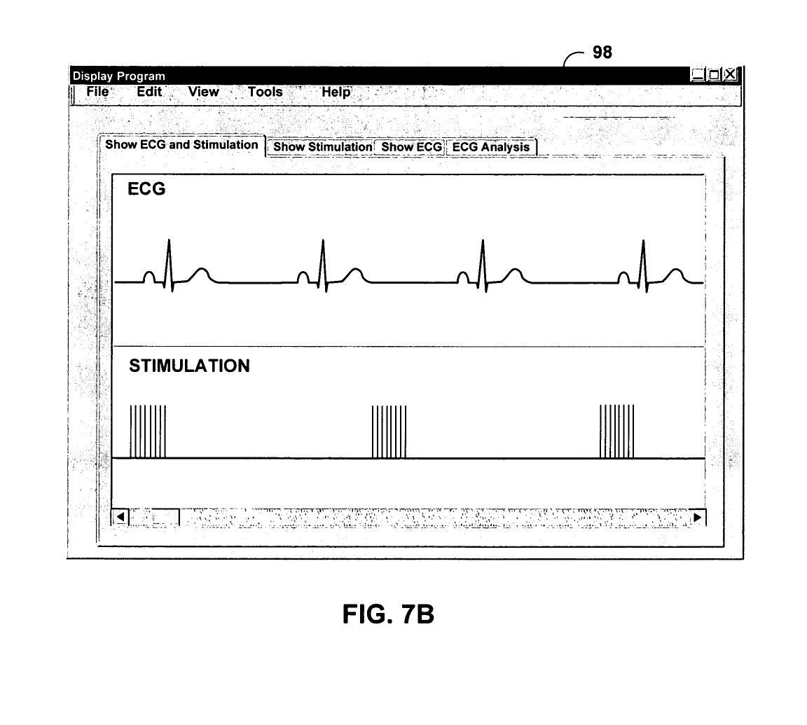

FIG. 7A is an exemplary screen illustration depicting an example report 96 displaying ECG information as viewed on a user interface. For example, report 96 may be viewed by a clinician of patient 16 on web browser 70A of FIG. 3 or on a display associated with external module 14. In particular, report 96 shows an ECG signal recorded by implantable stimulator 12. The clinician may analyze the ECG signal to determine whether patient 16 shows any signs of cardiac problems, abnormalities, or disease. The clinician may also analyze trend reports showing ECG information over longer time periods. The clinician may make recommendations to patient 16 based on the analysis of the ECG information. For example, the clinician may determine based on the ECG information that patient 16 is an appropriate candidate for an implantable cardiac device. In some embodiments, report 96 may be viewed by patient 16, or a family member or caregiver of patient 16.

FIG. 7B is an exemplary screen illustration depicting an example report 98 displaying ECG information and stimulation information as viewed on a user interface. As in FIG. 7A, report 98 may be viewed by a clinician, patient 16, or a family member or caregiver of patient 16, on web browser 70A or a display of external module 14. Report 98 allows a user to see the ECG information in the context of gastrointestinal stimulation therapy that is applied. Report 98 may aid the user in understanding any effects of stimulation therapy on the operation of the heart. Such information may be displayed in an embodiment in which both sensing intervals and stimulation intervals occur in an overlapping or simultaneous manner, as opposed to occurring in alternating intervals.

Various embodiments of the invention have been described. These and other embodiments are within the scope of the following claims.

1. A method comprising:
delivering electrical stimulation to a gastrointestinal tract of a patient via an implantable stimulator; and
sensing cardiac signals via a plurality of electrodes coupled to the implantable stimulator.

2. The method of claim 1, further comprising storing information based on the cardiac signals in a memory associated with the implantable stimulator.

3. The method of claim 1, further comprising transmitting information based on the cardiac signals from the implantable stimulator to an external module via a telemetry interface associated with the implantable stimulator.

4. The method of claim 3, further comprising displaying the information based on the cardiac signals via the external module.

5. The method of claim 1, further comprising:
analyzing information based on the cardiac signals; and
generating a cardiac therapy recommendation based on the analysis.

6. The method of claim 5, wherein generating a cardiac therapy recommendation comprises recommending that the patient be implanted with a cardiac device.

7. The method of claim 5, wherein generating a cardiac therapy recommendation comprises activating an implanted alert module.

8. The method of claim 7, wherein activating an implanted alert module comprises alerting the patient using one of an audio transducer, vibration in the implantable device, or stimulation by the implanted device of patient tissue.

9. The method of claim 5, wherein analyzing information comprises detecting based on the cardiac signals one of atrial fibrillation, ventricular fibrillation, atrial tachycardia, ventricular tachycardia, bradycardia, wide QRS complexes due to unsynchronized ventricular contractions, ectopic events, ischemic events, asystole, ST elevation, premature atrial contraction, bundle branch block, irregular heart rate, long QT syndrome, chronotropic incompetence, and myocardial infarction.

10. The method of claim 9, further comprising initiating an alert procedure upon detecting one of ventricular fibrillation and fast ventricular tachycardia based on the cardiac signals.

11. The method of claim 10, wherein initiating an alert procedure comprises issuing a communication to an emergency response system.

12. The method of claim 1, wherein sensing cardiac signals comprises sensing the cardiac signals via a plurality of electrodes, wherein at least one electrode extends away from the implantable stimulator via a lead.

13. The method of claim 1, wherein sensing cardiac signals comprises sensing the cardiac signals via a plurality of electrodes, wherein at least one electrode is disposed on a housing of the implantable stimulator.

14. The method of claim 1, further comprising alternating delivery of electrical stimulation to the gastrointestinal tract with sensing intervals of sensing the cardiac signals.

15. An implantable device comprising:
a plurality of electrodes;
a pulse generator that delivers electrical stimulation to a gastrointestinal tract of a patient via at least some of the electrodes; and
a processor that senses cardiac signals via at least some of the electrodes.

16. The device of claim 15, further comprising a memory that stores information based on the cardiac signals.

17. The device of claim 15, further comprising a telemetry interface that transmits information based on the cardiac signals to an external module.

18. The device of claim 15, wherein the processor analyzes information based on the cardiac signals and generates a cardiac therapy recommendation based on the analysis.

19. The device of claim 18, wherein the processor activates an implanted alert module based on the analysis.

20. The device of claim 15, wherein the sensed cardiac signals are electrocardiogram (ECG) signals.

21. The device of claim 15, wherein the plurality of electrodes comprises at least one electrode that extends away from the implantable device via a lead.

22. The device of claim 15, wherein the plurality of electrodes comprises at least one electrode that is disposed on a housing of the implantable stimulator.

23. A system comprising:
an implantable device that delivers electrical stimulation to a gastrointestinal tract of a patient with a pulse generator and senses cardiac signals via a plurality of electrodes; and
an external module, wherein the implantable device transmits information based on the cardiac signals to the external module via a telemetry interface associated with the implantable device.

24. The system of claim 23, further comprising a network server, wherein the external module transmits information based on the cardiac signals to the server and the server transmits the information to one or more network clients for viewing via a web browser, and wherein the web browser displays the information on a web page.

25. The system of claim 24, wherein the information based on the cardiac signals is displayed via one of the external module and the web page.

26. The system of claim 24, wherein the server analyzes information based on the cardiac signals and generates a cardiac therapy recommendation based on the analysis.

27. The system of claim 23, wherein the external module analyzes information based on the cardiac signals and generates a cardiac therapy recommendation based on the analysis.

28. The system of claim 23, wherein the external module is one of a patient monitor, a patient programmer, a physician programmer, a cellular telephone, a wristwatch, a personal digital assistant (PDA), and a pager.

29. An implantable device comprising:
means for delivering electrical stimulation to a gastrointestinal tract of a patient via an implantable stimulator; and
means for sensing cardiac signals via a plurality of electrodes coupled to the implantable stimulator.

30. A computer-readable medium comprising instructions that cause a programmable processor to:
deliver electrical stimulation to a gastrointestinal tract of a patient via an implantable stimulator; and
sense cardiac signals via a plurality of electrodes coupled to the implantable stimulator.
